SEQUENCE LISTING (27 sequences)

SEQ ID NO: 1 - RNA, 22 nt, Homo sapiens
  source 1..22: mol_type = other RNA
tgaggtagtaggttgtatggtt

SEQ ID NO: 2 - RNA, 22 nt, Homo sapiens
  source 1..22: mol_type = other RNA
tgaggtagtagtttgtgctgtt

SEQ ID NO: 3 - RNA, 23 nt, Homo sapiens
  source 1..23: mol_type = other RNA
taaagtgcttatagtgcaggtag

SEQ ID NO: 4 - RNA, 22 nt, Homo sapiens
  source 1..22: mol_type = other RNA
tagcttatcagactgatgttga

SEQ ID NO: 5 - RNA, 22 nt, Homo sapiens
  source 1..22: mol_type = other RNA
ttcaagtaatccaggataggct

SEQ ID NO: 6 - RNA, 21 nt, Homo sapiens
  source 1..21: mol_type = other RNA
ttcaagtaattcaggataggt

SEQ ID NO: 7 - RNA, 21 nt, Homo sapiens
  source 1..21: mol_type = other RNA
ttcacagtggctaagttccgc

SEQ ID NO: 8 - RNA, 19 nt, Homo sapiens
  source 1..19: mol_type = other RNA
ggggcctggcggtgggcgg

SEQ ID NO: 9 - RNA, 23 nt, Homo sapiens
  source 1..23: mol_type = other RNA
tagcaccatttgaaatcagtgtt

SEQ ID NO: 10 - RNA, 23 nt, Homo sapiens
  source 1..23: mol_type = other RNA
tcaagagcaataacgaaaaatgt

SEQ ID NO: 11 - RNA, 20 nt, Homo sapiens
  source 1..20: mol_type = other RNA
ggcggcggcggaggcggggg

SEQ ID NO: 12 - RNA, 22 nt, Homo sapiens
  source 1..22: mol_type = other RNA
caatcagcaagtatactgccct

SEQ ID NO: 13 - RNA, 22 nt, Homo sapiens
  source 1..22: mol_type = other RNA
tggcagtgtcttagctggttgt

SEQ ID NO: 14 - RNA, 22 nt, Homo sapiens
  source 1..22: mol_type = other RNA
caatcactaactccactgccat

SEQ ID NO: 15 - RNA, 23 nt, Homo sapiens
  source 1..23: mol_type = other RNA
taggcagtgtcattagctgattg

SEQ ID NO: 16 - RNA, 22 nt, Homo sapiens
  source 1..22: mol_type = other RNA
aatcactaaccacacggccagg

SEQ ID NO: 17 - RNA, 23 nt, Homo sapiens
  source 1..23: mol_type = other RNA
aggcagtgtagttagctgattgc

SEQ ID NO: 18 - RNA, 22 nt, Homo sapiens
  source 1..22: mol_type = other RNA
acgggttaggctcttgggagct

SEQ ID NO: 19 - RNA, 22 nt, Homo sapiens
  source 1..22: mol_type = other RNA
tcacaagtcaggctcttgggac

SEQ ID NO: 20 - RNA, 22 nt, Homo sapiens
  source 1..22: mol_type = other RNA
tccctgagaccctaacttgtga

SEQ ID NO: 21 - RNA, 22 nt, Homo sapiens
  source 1..22: mol_type = other RNA
gctacttcacaacaccagggcc

SEQ ID NO: 22 - RNA, 22 nt, Homo sapiens
  source 1..22: mol_type = other RNA
gctatttcacgacaccagggtt

SEQ ID NO: 23 - RNA, 23 nt, Homo sapiens
  source 1..23: mol_type = other RNA
agctggtgttgtgaatcaggccg

SEQ ID NO: 24 - RNA, 22 nt, Homo sapiens
  source 1..22: mol_type = other RNA
tggaatgtaaggaagtgtgtgg

SEQ ID NO: 25 - RNA, 22 nt, Homo sapiens
  source 1..22: mol_type = other RNA
acagcaggcacagacaggcagt

SEQ ID NO: 26 - RNA, 22 nt, Homo sapiens
  source 1..22: mol_type = other RNA
tccagcatcagtgattttgttg

SEQ ID NO: 27 - RNA, 22 nt, Homo sapiens
  source 1..22: mol_type = other RNA
aacaatatcctggtgctgagtg

(19) United States
(12) Patent Application Publication
(10) Pub. No.: US 2023/0277596 A1
(43) Pub. Date: Sep. 7, 2023

(54) EXOSOME COMPOSITION AND METHOD OF MANUFACTURE

(71) Applicant: Vivex Biologics Group, Inc., Atlanta, GA (US)

(72) Inventors: Connie Chung, Miami, FL (US); Shabnam Namin, Miami, FL (US); Timothy Ganey, Tampa, FL (US); Renaud Sicard, Miami, FL (US)

(21) Appl. No.: 18/051,663

(22) Filed: Nov. 1, 2022

Related U.S. Application Data
(63) Continuation of application No. 16/710,472, filed on Dec. 11, 2019.
Application No. 16/710,472 is a continuation-in-part of application No. 16/983,190, filed on Aug. 3, 2020.
Application No. 16/983,190 is a continuation of application No. 14/810,003, filed on Jul. 27, 2015, now Pat. No. 11,077,147.
(60) Provisional application No. 62/794,912, filed on Jan. 21, 2019.
(60) Provisional application No. 62/194,574, filed on Jul. 20, 2015.

(51) Int. Cl. A61K 35/28; A01N 1/02; A61K 9/19; A61K 35/32

(52) U.S. Cl. CPC A61K 35/28; A01N 1/0221; A61K 9/19; A61K 35/32

(57) ABSTRACT

Compositions of exosomes are provided that include a plurality of exosomes and a biocompatible cryoprotectant, such that the exosomes are suspended in the biocompatible cryoprotectant as a colloidal suspension of exosomes. Preferably, the cryoprotectant is a carboxylated E-poly-l-lysine (COOH-PLL) cryoprotectant, but other moieties might be extended to the claim of hybridization polymers to incorporate preferred embodiments for lineage and tissue specific intentions. The colloidal suspension of exosomes can be frozen at −65 degrees C. or colder and thereafter stored as a frozen composition of exosomes or can be freeze-dried and thereafter stored at ambient conditions in a vacuum sealed container. Also provided are kits comprising the composition of exosomes and methods of making the compositions of exosomes.

### RELATED APPLICATIONS

The present invention is a continuation of co-pending application Ser. No. 16/710,472 filed on Dec. 11, 2019, which claims benefit of priority from provisional application No. 62/794,912 filed on Jan. 21, 2019. The present invention is also a continuation-in-part of co-pending application Ser. No. 16/983,190 filed on Aug. 3, 2020, which is a continuation of application Ser. No. 14/810,003 filed on Jul. 27, 2015, now U.S. Pat. No. 11,077,147, which claims benefit of priority from provisional application No. 62/194,574 filed on Jul. 20, 2015.

### SEQUENCE LISTING XML

The miRNA sequences in the 24 KB XML file Sequence Listing with file name: DN0345CONTseq.xml attached are incorporated by reference, the Sequence Listing was created Mar. 21, 2023 from FIGS. 21 and 22 taken from data in the in depth analysis report of Sep. 24, 2018. The sequences are described in the sequence listing herein attached and depicted after the detailed description.

### TECHNICAL FIELD

The disclosure provides a composition of exosomes and a method of manufacturing the composition of exosomes.

### BACKGROUND OF THE INVENTION

The use of stem cells in compositions for use in therapeutic treatments has been commonly accepted. Maintaining the viability of these cells from recovery to processing and storage has been a challenge. Various cryoprotectants have been used to preserve the cells. Most, like DMSO and other glycerol-based products, require the protectant to be washed away prior to implanting the cells. This often leads to a significant loss of viable cells available from the initial amount. Accordingly, the outcomes for patients can vary widely.

In U.S. Pat. No. 9,675,643, a way to protect the cell was discovered using a polyampholyte carboxylated ε-poly-l-lysine based protectant suitable for direct implantation without washing.

In a related patent, U.S. Pat. No. 9,687,511, it was discovered such a protectant could be used to protect acellular compositions.

The following compositions and methods described herein form the basis of the present invention.

### SUMMARY OF THE INVENTION

In certain embodiments of the present invention, a new method and composition has been developed that employs exosomes that preferably are treated with DMSO free protectants that can be used, stored, frozen or even freeze-dried and stored at ambient temperature while maintaining an ability to stimulate differentiation of primitive cells such as mesenchymal stem cells. Importantly, the exosomes can be tuned to exhibit different cell stimulating properties to enhance their performance when implanted.

A composition of exosomes has a plurality of exosomes and a biocompatible cryoprotectant. The exosomes are suspended in the cryoprotectant creating a colloidal suspension of exosomes. Biocompatible as used herein is defined as being DMSO free and or not requiring washing before therapeutic use. The plurality of exosomes is in a concentration of 1×10³ to 1×10¹², is preferably greater than 1×10⁸ exosomes/ml. This concentration can be made higher or lower per ml based on the manufacturer's choice. The colloidal suspension of exosomes can be frozen at −65 degrees C. or colder forming a frozen composition of exosomes or is freeze-dried forming a freeze-dried composition of exosomes. The freeze-dried composition of exosomes can be stored at ambient conditions in a vacuum sealed container. The freeze-dried exosomes are preferably at a concentration of 8E8. Preferably, the cryoprotectant is a carboxylated ε-poly-l-lysine (COOH-PLL) cryoprotectant.

The carboxylated ε-poly-l-lysine (COOH-PLL) provides stability to exosome dispersion by resisting flocculation or agglomeration. The cryoprotectant can also be configured to induce an electrostatic charge stabilization. The cryoprotectant can be tuned to an acidic pH, a neutral pH or a base pH to achieve positive, negative or zero zeta potentials. A 7.0 pH is neutral yielding a 0 mV zeta potential. The composition of claim 6 wherein the carboxylated ε-poly-l-lysine (COOH-PLL) cryoprotectant can be configured to yield a positive or negative mV zeta potential in the composition of exosomes. Alternatively, the carboxylated ε-poly-l-lysine (COOH-PLL) cryoprotectant can be configured to yield a˜10 mV zeta potential.

The carboxylated ε-poly-l-lysine (COOH-PLL) cryoprotectant creates a steric stabilization or repulsion by coating polymers on surfaces of the exosome particles preventing the particles from coming into contact with each other. The thickness of the coating is sufficient to keep particles separated by steric repulsions between the polymer layers. The cryopreserved and lyophilized exosomes in the carboxylated epsilon PLL cryoprotectant enhance regenerative capabilities. The freeze-drying maintains biological properties of the exosomes in terms of osteoinduction. Exosomes within the composition retain biological protein markers CD63, CD9 and CD81 in the composition, which are known canonical exosome markers and MSC negative control marker SSEA-4. Also, the exosomes exhibit distinct miRNA profiles; regulation of essential miRNA to control biological processes such as osteogenesis and angiogenesis, wherein examples of negative regulators that are downregulated in our final lyophilized exosome product: miR-125, miR-214 and wherein examples of positive regulators that are upregulated in our final lyophilized exosome product: let-7c, let-7i, miR-21, miR-26a, miR-27a, miR-335, miR-3960.

A kit with an exosome composition can be made having a quantity of exosomes and a volume of cryoprotectant, wherein the quantity of exosomes is suspended in the cryoprotectant creating a suspension of exosomes which is frozen or freeze-dried to form an exosome composition and a quantity of one or more bone derived components or combinations thereof of a bone gel, a cortical bone, a cancellous bone, or a demineralized bone, a partially mineralized bone or a mineralized bone. The kit has the exosome composition combined with one or more of the bone derived components to form an osteoinductive or osteoconductive product. The bone derived components are fibers, or particles, or combinations thereof.

A method of making a composition of exosomes has the steps of: a) creating a solution from a washed bulk tissue source submerged with at least twice the volume of prepared Processing Media with Antibiotics (PMWA) and incubating at an elevated temperature over a duration of several hours, the solution containing a mixture of non-whole cellular components and whole cells; b) filtering the solution from the washed bulk tissue source; c) separating the non-whole cellular components from the whole cells by centrifugation forming a cell pellet and a supernatant above the cell pellet; d) ultra-centrifuging the supernatant to form a pellet of cell debris and discarding the pellet of cell debris; e) filtering the supernatant with a sub-micron filter; and f) ultracentrifuging the supernatant to form an exosome pellet. The duration of incubation can vary from as few as one hour up to 24 hours or more at the elevated temperature. The duration of incubation of several hours preferably is between 18-24 hours. The sub-micron filter is preferably 0.5 micron or less, more preferably 0.4 micron or less, most preferably a 0.2-micron filter.

The method of claim 24 further has the steps of: suspending the exosome pellet in a fluid wash; ultracentrifuging to form the exosome pellet and a second supernatant with unwanted proteins; and discarding the second supernatant. The method further has the step of forming an exosome fluid suspension by suspending the exosome pellet in a fluid. The method further has the step of measuring a sample quantity of exosomes in the suspended fluid to establish a quantity of exosomes per ml. The method further has the step of: freeze drying the exosomes in the fluid, wherein the fluid is preferably a cryoprotectant, but can include saline or DPBS solution. Alternatively, the exosome fluid suspension can be frozen forming a frozen liquid for storage.

### Definitions

Biocompatible as used herein does not require washing before therapeutic use of the composition. Alternatively, DMSO-free or something similar could be used.

DNase—Deoxyribonuclease is any enzyme that catalyzes the hydrolytic cleavage of phosphodiester linkages in the DNA backbone, thus degrading DNA.

DMEM, DMEM/LG—Dulbecco's Modified Eagle Medium, low glucose. Sterile, with: Low Glucose (1 g/L), Sodium Pyruvate; without: L-glutamine, HEPES (4-(2-hydroxyethyl)-1-piperazineethanesulfonic acid).

DMSO—Dimethyl sulfoxide (DMSO) is an organosulfur compound with the formula (CH₃)₂SO. This colorless liquid is an important polar aprotic solvent that dissolves both polar and nonpolar compounds and is miscible in a wide range of organic solvents as well as water.

DPBS—Dulbecco's Phosphate Buffered Saline.

CBT-MIXER—Mixing blade for Cancellous Bone Tumbler Jar.

Cold Media—Media used during the preparation of vertebral bodies for initial processing.

Cryopreserved—Tissue frozen with the addition of, or in a solution containing, a cryoprotectant agent such as glycerol, or dimethylsulfoxide, or carboxylated poly-l-lysine.

“E” stands for the word “exponent” in scientific notation, it is used to specify how many places to the right to move the decimal point of the number that comes before it. 5E6 is the number 5,000,000, for example. The way to say the number is, “five times ten raised to the sixth power”. It's basically a form of shorthand that means 5*10⁶. Sometimes the number after E can be negative. For example, 5E-6 would then specify how many places to the left to move the decimal point. In this case the number is 0.000005.

Freeze-dried/Lyophilized as used herein are used interchangeably—Tissue dehydrated for storage by conversion of the water content of frozen tissue to a gaseous state under vacuum that extracts moisture.

Normal Saline—0.9% Sodium Chloride Solution.

Packing Media—Media used during initial processing and storage of the processed vertebral bodies prior to bone decellularization.

PBS—Phosphate Buffered Saline.

Processing Media—Media used during bone decellularization that may contain DMEM/Low Glucose no phenol red, Human Serum Albumin, Heparin, Gentamicin and DNAse.

### BRIEF DESCRIPTION OF THE DRAWINGS

The patent or application file contains at least one drawing/photograph executed in color. Copies of this patent or patent application publication with color drawing(s) will be provided by the Office upon request and payment of the necessary fee.

The invention will be described by way of example and with reference to the accompanying drawings in which:

FIG. 1 shows a photograph of a cut vertebral body taken from a spine of a cadaver donor.

FIG. 2 shows a photograph of the vertebral body after being cut into cubic pieces and immersed in a packing media.

FIG. 3 shows a photograph of the bulk bone material after being ground and immersed in packing media and placed in a jar for later tumbling.

FIG. 4 is a flowchart depicting the phase 1 process.

FIG. 5 is a flowchart depicting the phase 2 process.

FIG. 6 is a flowchart depicting the packaging process including freezing and final packaging of frozen liquid configuration.

FIG. 7 is a flowchart depicting the freeze-drying process including freeze-drying for freeze-dried configuration.

FIG. 8 is a flowchart depicting the final packaging and storage process for freeze-dried configuration.

FIG. 9 is a photograph of the exosome composition of the present invention.

FIG. 10 is a photograph of the exosome composition of the present invention packaged in a jar and in a vial.

FIG. 11 is a graph showing exosome flow cytometry marker analysis.

FIG. 12 is a graph showing osteoinductive testing results.

FIG. 13 is a graph and pictures showing human mesenchymal stem cell (hMSC) uptake results.

FIG. 14 shows a moldable bone gel.

FIG. 15 shows Scanning Electron Microscope (SEM) images of spine derived exosomes.

FIG. 16 is a graph showing spine derived exosome flow cytometry marker analysis.

FIG. 17 shows graphs of control and test groups for osteoinductive testing results with exosomes resuspended in DPBS.

FIG. 18 shows graphs of exosomes/batch, #units/batch and exosomes/cell.

FIG. 19 shows graphs of control and test groups for ZetaView exosome titration (n=2).

FIG. 20 is a figure depicting COOH-epsilon PLL coated exosome in neutral pH environment.

FIG. 21 is a table of miRNA positive regulators.

FIG. 22 is a table of miRNA negative regulators.

FIG. 23 is a graph of flow cytometry results—combined donors (n=3).

### DETAILED DESCRIPTION OF THE INVENTION

With reference to the present invention which is a composition of exosomes derived from a tissue source, the composition can be freeze-dried and stored at ambient conditions or frozen for storage. Preferably, in either condition, the exosomes are intermixed with a cryoprotectant that is non-DMSO based, preferably a carboxylated ε-poly-l-lysine cryoprotectant.

While it is understood the exosome composition can be derived from any number of tissue sources, such a muscle, fat, organs or bone or bone marrow, the representative examples and test data are based on exosomes derived from bone marrow from a cadaver donor.

The composition is directed to achieving a concentration of exosomes from the source tissue. The source tissues have been markedly similar to those wherein successful harvesting of stem cells has been accomplished. These include, by way of example, placental tissues, bone marrow, umbilical cords, whole blood, and fat. The harvesting of exosomes yields compositions rich in concentrations of exosomes, typically concentrations are 1E6 to 1E10 per ml. It has further been determined that these concentrations of exosomes can be combined with other materials to facilitate delivery and use in medical procedures. By way of example, kits with the concentrated exosome composition when made having other vials or containers of bone particles or bone fibers that are mineralized or demineralized which are mixed to form an exosome laden bone blend for use in bone repair. Similarly, the concentrated exosome concentration having separate vials of nucleus pulposus particles provided as a kit when mixed together yield a regenerative spinal disc repair composition. Kits of vials of cartilage material or other soft tissue provide unique combinations when Mixed with the concentrated exosomes that are particular useful in repairing such tissue tears such as knee injuries and Achilles tendon tears, particularly so when the tear is only partial. These uses are in no way intended to be limiting, but rather exemplary of a wide range of uses either singularly or combined with other tissue types in the form of a kit. One particularly useful combination is a concentration of exosomes loaded in a bone gel. Bone gels can be in the form of a moldable gel or paste or can be a cohesive blend of gel and bone particles and are available in a range of types perfectly suited to be loaded with a concentration of exosomes. This combination ensures the exosomes are delivered directly to the bone repair site. As noted, these combinations yield a remarkable performance gain in remodeling and regenerating damaged tissue. Fabrication of a variety of types include molding, forming, drying, centrifugal casting, cryo-lyophilization and use at varying dehydrated states and carrier combinations to sustain malleability and to print in additive investments of specific shape and volume, commonly called 3D printing. all of which can be used with the compositions of exosomes and biocompatible cryoprotectant. This is particularly convenient in the dehydrated or freeze-dried condition wherein the composition can be built into bioabsorbable carriers allowing for a concentration of exosomes to be effectively time released.

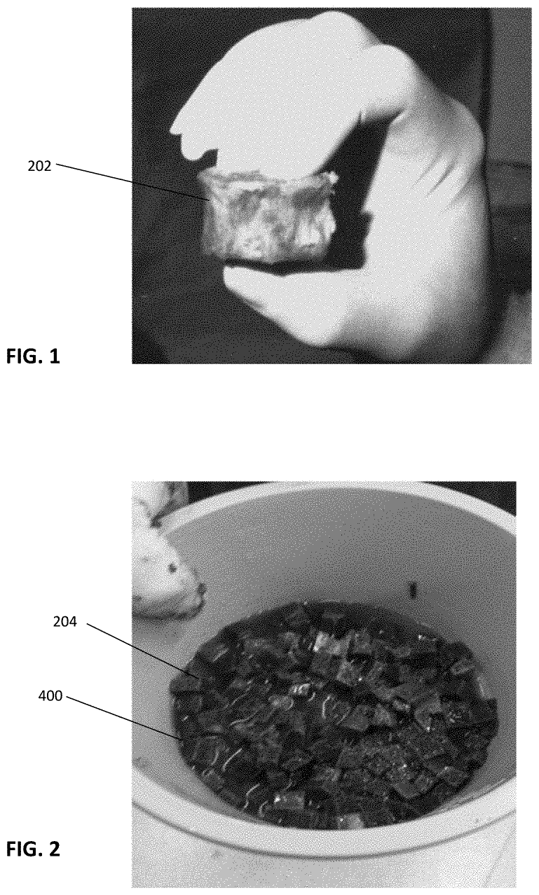

With reference to certain embodiments of the present invention which is a tissue regenerative biological composition 100 made from bone marrow 200, it is believed best understood by the methods used to process and recover the biological composition, as illustrated in the FIGS. 1-3.

The first steps are to collect, recover and process bone marrow 200 from a cadaver donor. To do this, the spine is removed aseptically from the cadaver and the resultant spine segment is covered by cold media. The cold media has 0.5 ml of Heparin; 10,000 units/ml per 500 ml of DMEM. DMEM is a sterile solution with low glucose (1 g/L), Sodium Pyruvate; without L-glutamine, or HEPES. This cold media is used for packaging the spine segments for later processing. At this point, the spine segment includes a plurality of vertebral bodies 202.

Processing of the spine-derived exosomes was conducted at VIVEX Biomedical, Inc. facilities and consists of two phases. During processing, minimal manipulation is used to ensure the basic function(s) of the tissue are not compromised and to ensure the native state of the cellular tissue will remain intact with no events of expansion performed. During Phase I of processing, the exosomes are exposed to Acid-Citrate-Dextrose, Solution A (ACD-A), DMEM base media, HSA Albumin 25%, DNAse, Heparin, antibiotics, Collagenase, 0.9% Sodium Chloride, PBS and DPBS. During Phase II of processing, exosomes are purified from the supernatant by differential ultracentrifugation and ultrafiltration. There are two available final configurations of the exosome product: (1) Frozen Liquid; (2) Freeze-Dried. Frozen configuration will be resuspended in VIA COAT™, a DMSO-free carboxylated ε-poly-l-lysine cryoprotectant. The liquid exosome suspension will be aliquoted into cryovials and packaged in a tear pouch within another peel pouch and stored at ≤65° C. For preparing the freeze-dried configuration, the liquid exosome suspension will be aliquoted into amber serum vials and undergo freeze-drying. Following freeze-drying, the dried exosome product will be final packaged in a tear pouch within another peel pouch and stored at ambient temperature. The expected shelf-life for both configurations is two (2) years from the final packaging date.

Once the spine is recovered, it is placed in Cold Media solution (DMEM media and Heparin) until it is ready for further processing. The ACD-A solution is then prepared by combining 1000 mL of 0.9% Sodium Chloride and 118 mL of ACD-A into a sterile bottle. Using a scalpel and/or forceps, excess soft tissue surrounding the spine is removed. The vertebral bodies (VBs) are then excised using a band saw and submerged into the ACD-A solution. The VBs are then cut into smaller pieces (approximately 1 cm³) using the band saw. These smaller pieces are also kept submerged in the ACD-A solution. The bone pieces are then ground to 4-10 mm pieces using a bone grinder. The ACD-A solution is used to assist with this process until the final Bulk Cortical-Cancellous crush component is acquired.

The clinical technician must remove as much soft tissue as possible and cut each vertebral body 202 with a saw. These vertebral bodies 202, once cleaned, of all adherent soft tissue around the cortical surfaces will look as shown in FIG. 1.

Once a cleaned vertebral body 202 is obtained, the next step involves cutting each vertebral body 202 into pieces, each piece 204 roughly 1 cm³. The cut pieces 204 being immersed in a packing media 400. The exemplary packing media can be DMEM with 0.5 ml Heparin and 1.25 ml of DNAse added.

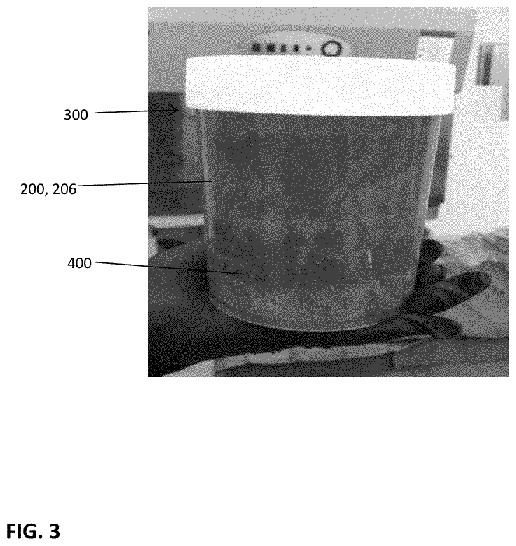

Once all the vertebral bodies 202 have been cut, the pieces 204 are transferred to a bone grinder. The bone is ground into 4-10 mm pieces using packing media 400 to facilitate the grinding process. The ground bone 206 (bulk cortical-cancellous crushed) and all of the packing media 400, estimated volume of 500 ml are transferred into ajar 300 where 0.5-1.0 ml of Gentamicin is added to the jar 300 with ground bone 206 and packing media 400. At this point, the crushed bone 206, including cellular soft marrow 200, is intermixed as shown in FIG. 3.

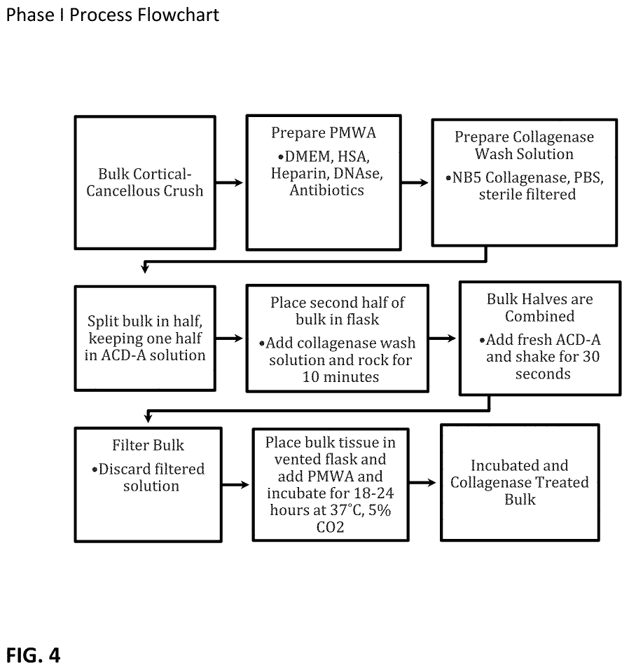

Once the Bulk Cortical-Cancellous Crush is produced, Phase I of the manufacturing process begins, depicted in the diagrammatic flowchart of FIG. 4. Processing Media with Antibiotics (PMWA) is prepared by combining 447.25 mL of DMEM media, 50 mL of HSA Albumin 25%, 0.5 mL of Heparin, 1 mL of Gentamicin, and 1.25 mL of DNAse. At this time, the Collagenase Wash solution is also prepared by combining 250 mg of NB5 Collagenase and 250 mL of warm PBS into a sterile glycerol bottle. Once the Collagenase Wash solution is mixed thoroughly, it is then sterile filtered with a 0.2 μm PES membrane filter unit. It is understood the filter pore size could be less than 0.5 micron, or less than 0.4 micron, but 0.2 micron is preferred as it isolated out larger unneeded debris from the exosomes. The weight of the Bulk Cortical-Cancellous Crush is measured, and half of the bulk is left in the ACD-A wash solution, while the other half is separated out into an Erlenmeyer flask with double the volume of the prepared Collagenase Wash solution. This flask is placed on a rocker for 10±1 minutes to provide a light agitation. The Collagenase Wash solution and bulk is then filtered and sieved through a 1 mm sieve, discarding any tissue less than 1 mm. This bulk portion of tissue is then combined back with the other bulk half. Fresh ACD-A solution is added to the total bulk and the container is vigorously shaken for approximately 30 seconds. The bulk is then filtered, and the wash solution is discarded. The washed bulk tissue is transferred into a sterile 1000 mL vented Erlenmeyer flask and submerged with at least twice the volume of the prepared PMWA. This flask is placed into an incubator at 37±2° C., 5% CO2 for 18-24 hours.

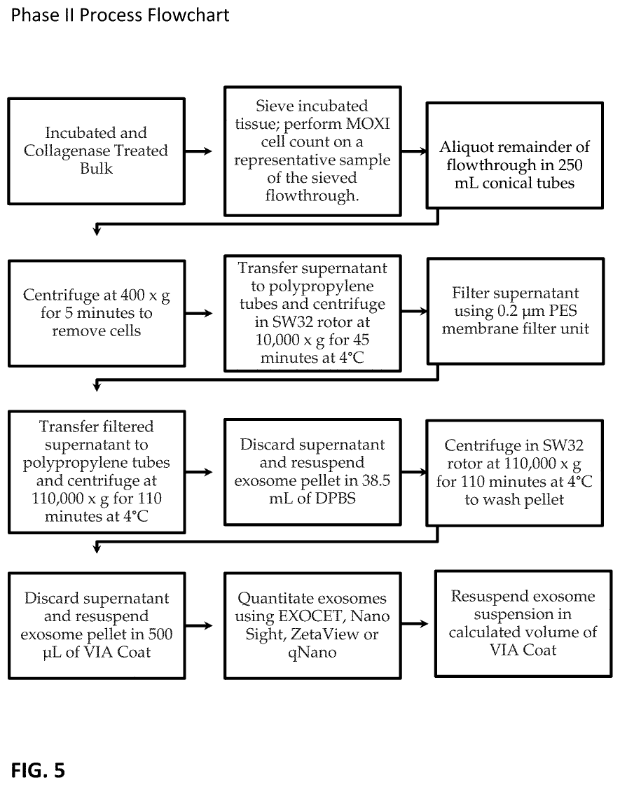

After the Bulk Cortical-Cancellous Crush incubation process is over, the tissue is ready for Phase II of the Spine-Derived Exosome manufacturing process. Phase II Process is depicted in the diagrammatic flowchart of FIG. 5. Using aseptic technique, the flask is removed from the incubator and all subsequent sample handling is performed under an ISO Class 5, Class II biological safety cabinet (BSC). The contents of the flask are filtered through a sieve stack. A representative sample of the filtered solution is taken and used to obtain a cell count using the automated MOXI Flow cell counter for the purpose of calculating total exosomes per live cell. The filtered solution is then aliquoted into conical tubes and centrifuged at 400×g for 5 minutes to pellet cells. The supernatant formed after centrifugation is removed without disturbing the pellet and aliquoted into sterile tubes designed to withstand the forces of ultracentrifugation, such as thin-walled open-top polypropylene tubes for differential ultracentrifugation. The tubes are loaded into sterile swinging buckets, which are then subsequently loaded in the SW32 rotor in the Beckman Optima XE-90 Ultracentrifuge. Alternatively, a fixed-angle rotor or equivalent ultracentrifuge may be used. The samples are centrifuged at 10,000×g for 45 minutes at 4° C. to pellet cell debris. Following pelleting of cell debris, the supernatant is removed and filtered with a 0.2 μm PES membrane filter unit using vacuum pressure. Other types and pore sized filters could be optionally used as long as the exosomes are effectively isolated and captured in the exosome pellet. The filtered supernatant is then aliquoted into new and sterile thin-walled open-top polypropylene tubes and centrifuged at 110,000×g for 110 minutes at 4° C. to pellet a crude exosome fraction. Following exosome pelleting, the supernatant is removed and discarded. The pellet is resuspended in 38.5 mL of DPBS and centrifuged again at 110,000×g for 110 minutes at 4° C. to wash the exosomes and remove contaminating proteins. The supernatant is then removed and discarded, and the exosome pellet is resuspended in 1 ml of VIA COAT™ cryoprotectant. Exosomes are quantified. Any number of measuring techniques can be employed such as using EXOCET Quantitation Kit (SBI), NANOSIGHT™ (Malvern), or qNANO (IZON™) methods. A more accurate technique involves measuring using a ZETAVIEW™ (Particle Metrix) instrument which is a nanoparticle tracking analyzer (NTA). Based on the exosome concentrations, the number of units (≥1×10⁸ exosomes/mL) that will be prepared is calculated. The exosome suspension is resuspended in an appropriate volume of VIA COAT™. The concentrations of exosomes can vary based on the techniques and harvesting protocols. Concentrations of these small nanoparticle sizes can be as low as 1E3 exosomes/ml to as high as 1E12 exosomes/ml or higher. The technique described above typically yields 1E8 or greater exosomes/ml. That said, the concentrations can be collected at very high concentrations and deliberately diluted in a fluid such as water, normal saline, blood or any other suitable biocompatible fluid to achieve lower concentrations if desired. This can be useful to lower the unit cost per dose and to provide a less concentrated dose. Accordingly, the composition can have a wide range of concentrations dependent on the application.

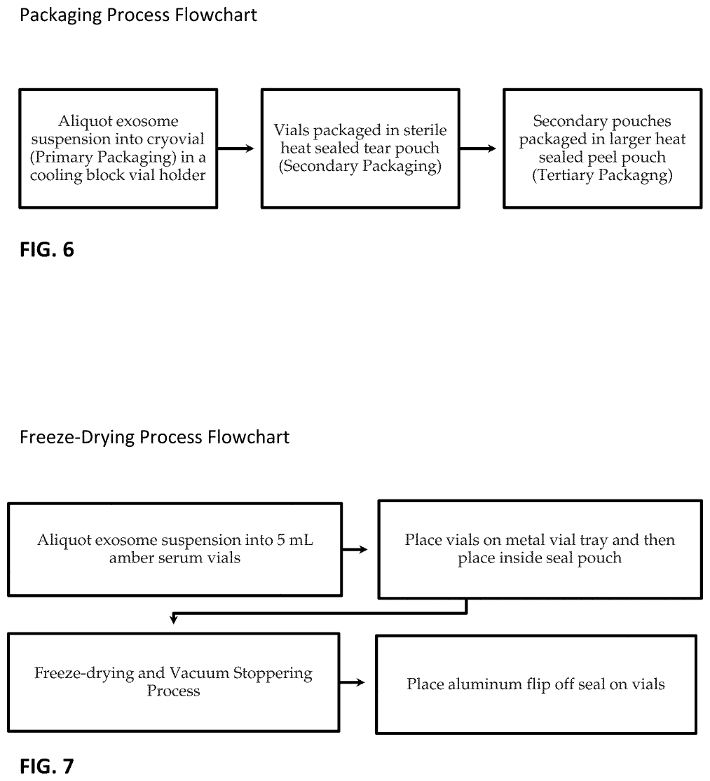

Freezing or Freeze-Drying and Final Packaging Process is depicted in the charts of FIGS. 6 and 7 respectively.

The required cryovials that will be used will be placed in cooled vial holders during the aliquoting process. The cryovials aliquoted with the exosome suspension are then capped and placed into a sterile secondary aluminum oxide polyester tear pouch and heat sealed. The inner pouch is then placed into a larger, tertiary sterile aluminum oxide polyester peel pouch and heat sealed. This tertiary pouch can then be treated as non-sterile. The packaged units are placed into a −65° C. or colder freezer overnight before transferring directly into the freezer for long term storage.

The Freeze-Dried Configuration is depicted in the chart of FIG. 7.

For samples undergoing freeze-drying, exosome suspension is aliquoted into 2 mL amber serum vials. Vials are placed on a metal vial tray, which is then placed into a sterile seal Tyvek pouch. The pouch is then loaded into a pre-cooled (to −40° C.) freeze-dryer to undergo a 29-hour freeze-drying cycle. Following completion of the freeze-drying cycle, vial stoppers and flip off 20 mm aluminum seals are attached to the vials. Sealed vials are then placed into a sterile secondary aluminum oxide polyester tear pouch and heat sealed. The inner pouch is then placed into a larger, tertiary sterile aluminum oxide polyester peel pouch and heat sealed. This tertiary pouch can then be treated as non-sterile.

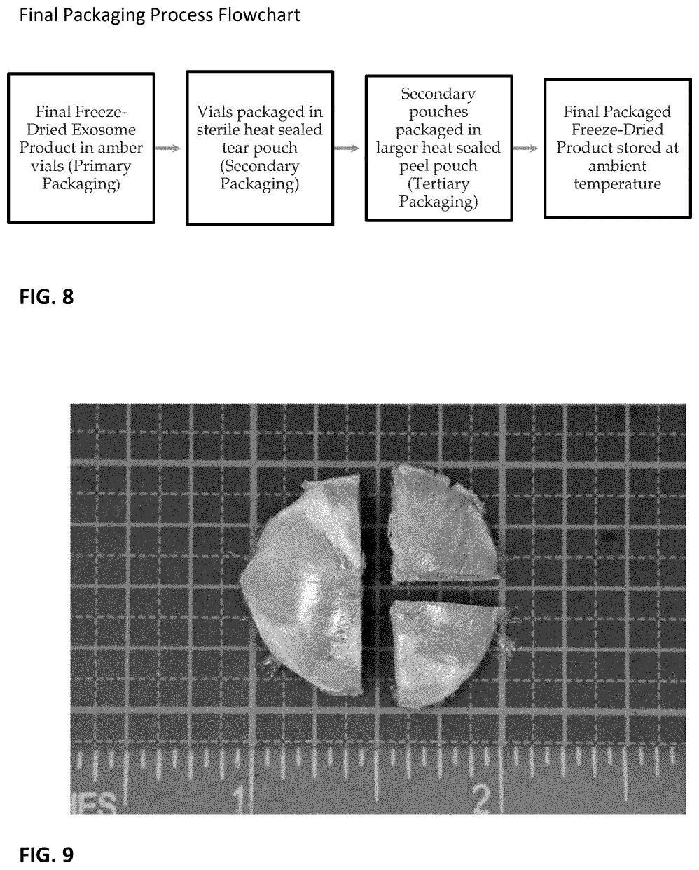

As shown in FIG. 8, the final freeze-dried exosome product is stored at ambient temperature until it is prepared for shipment. The current expiration date of both the frozen and freeze-dried configurations of the Spine Derived Exosome Product is 2 years from the date of freezing. These dates are likely to be much longer, and in the case of the freeze-dried product, may be stored indefinitely in their vacuum sealed containers.

FIG. 9 is a photograph of the exosome composition of the present invention. As shown in FIG. 9, the cryoprotected exosomes after freeze drying have a consistency that retains its shape. The material is sufficiently cohesive and moldable to any shape making it ideal for bone repair. As shown, a disk of the material has been cut into a semi-circle and two quarter circles and the pieces do not crumble but adhere together. This makes the exosomes ideal for not only packaging, but also providing clinicians with specific functional molds.

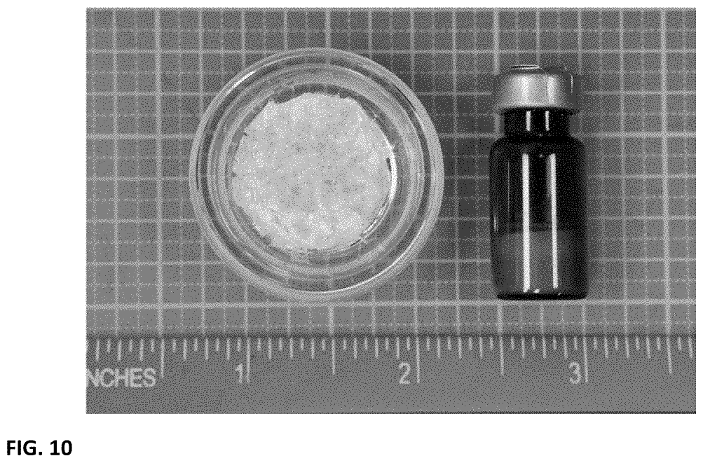

FIG. 10 is a photograph of the exosome composition of the present invention packaged in ajar and in a vial. In the jar, it has been noted that the exosomes stay in the confined space as a single mass and will slide inside the vial without separating into pieces.

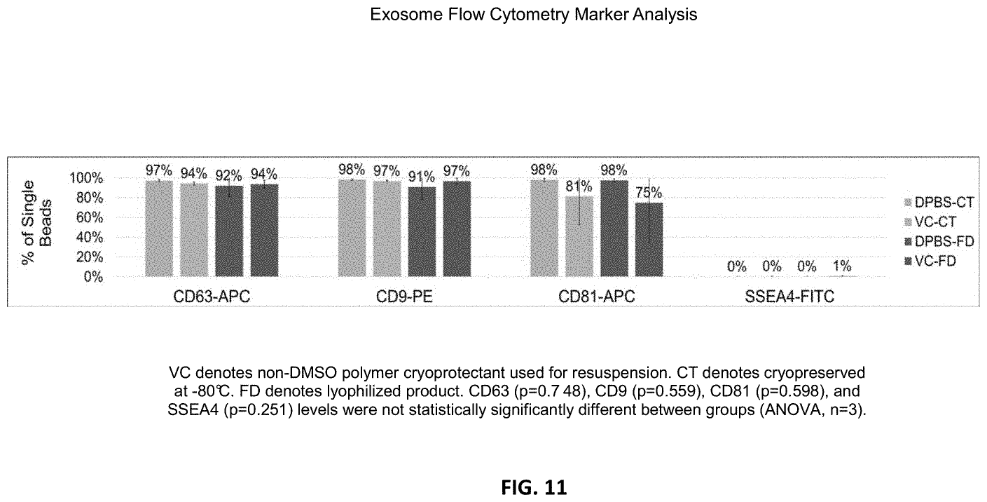

Cryoprotection with Non-DMSO Polymer Cryoprotectant and Lyophilization of Spine-Derived Exosomes Promote Osteoinduction and Mesenchymal Stem Cell Uptake is described in FIGS. 11-13.

Facilitating complete bone repair remains a major clinical challenge for orthopaedic surgeons. Much evidence has recently accumulated demonstrating the effectiveness of exosomes secreted by Mesenchymal Stem Cells (MSCs) in promoting bone regeneration. This cell-free therapeutic platform eliminates the current challenges faced with maintaining cellular viability in allograft transplantation as well as minimizes any risk of immunogenic effects. Studies addressing the optimization of exosome manufacturing and production for bone regenerative therapy are currently lacking in the field.

The inventors understood that exosomes are nanosized vesicles that function as critical mediators of intercellular communication and impart its therapeutic efficacy by cellular uptake mechanisms. Although exosome purification techniques have been extensively reported in the literature, cryopreservation and lyophilization recommendations are not well defined. Previously, the inventors uncovered the osteoinductive role of fresh spine-derived exosomes, not subject to cryopreservation or lyophilization. They sought to determine if cryoprotecting and lyophilizing these exosomes would still retain its biological properties and therapeutic benefits in the context of bone regeneration. This study allowed them to explore the possibility of providing clinicians with an “off-the-shelf” exosome formulation, eliminating the time and costs associated with handling a frozen biologic product. Confirming uptake in recipient hMSCs would also provide further evidence that lyophilized exosomes retain an ability to sustain intercellular communication.

The inventors hypothesized that cryopreserving and lyophilizing spine-derived exosomes will enhance osteoinductive and cellular uptake properties.

To test their hypothesis, they purified exosomes from qualified cadaveric human spines by ultrafiltration and subsequent differential ultracentrifugation of the clarified supernatant. Exosomes were resuspended in DPBS or a non-DMSO polymer cryoprotectant and either frozen at −80° C. or lyophilized. The expression of alkaline phosphatase, a widely accepted bone marker, was measured following treatment of C2C12 cells with 1×10⁹ or 2×10⁹ spine-derived exosomes. For the uptake assay, hMSC membranes were stained with CFDA and exosome membranes were stained with the lipophilic tracer Dil and subsequently purified by ExoQuick-TC (SBI) precipitation and centrifugation. CFDA-stained hMSCs were incubated with Dil-stained exosomes for various time points up to 24 hours and Dil incorporation in hMSCs was assessed by fluorescent microscopy and flow cytometry, see FIGS. 11-13.

OI (osteoinductive) testing revealed that cryoprotection and lyophilization of spine-derived exosomes retained biological function by significantly enhancing the release of alkaline phosphatase at both concentrations tested at levels surprisingly comparable to BMP-4 positive control. Levels were greater than produced by cells treated with exosomes in DPBS, suggesting the cryoprotection and lyophilization optimizes osteoinductive properties of exosomes. Further, the uptake assay revealed that more exosomes, cryoprotected and lyophilized, were taken up by hMSCs compared to DPBS exosomes, pointing to a potential mechanism for its enhanced osteoinductive capabilities. This study supported their goal of providing clinicians with an optimally formulated, convenient, safe and effective exosome product to repair injured tissue and restore bone function.

With reference to FIG. 11, Flow Cytometry with Latex Beads: 1. Aldehyde/Sulfate latex beads (4% w/v, 4 μm) coated with exosome sample. 2. Beads stained for exosomal flow markers CD63, CD81, and CD9 and MSC negative control marker SSEA-4. 3. Samples analyzed by flow cytometry and single beads gated for analysis.

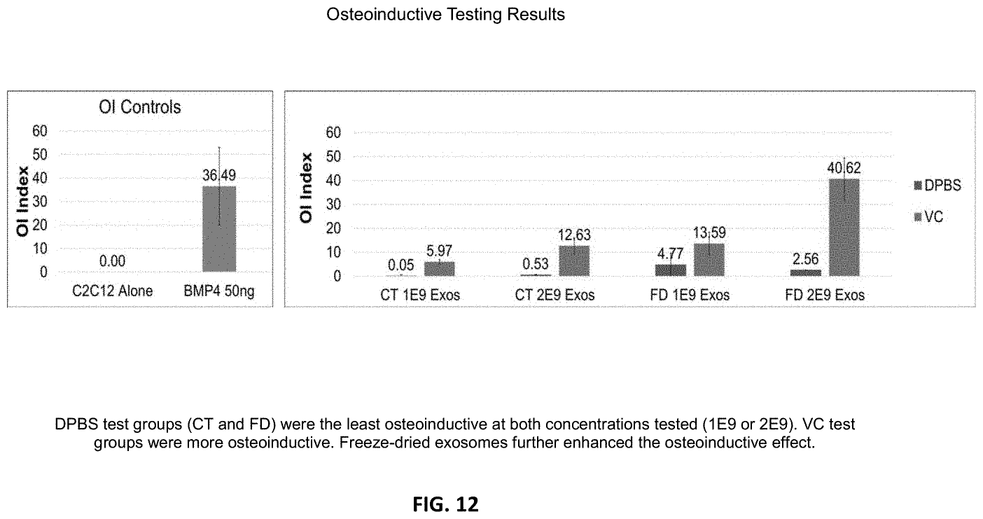

With reference to FIG. 12, Osteoinductive Testing with C2C12 (Pluripotent) Cells: 1. C2C12 cells seeded in 24-well plate. 2. Cells treated with different exosome sample types. BMP-4 treatment used for positive control. 3. Following 48-hour exposure, cells assayed for alkaline phosphatase activity using Thermo Scientific Pierce PNPP Substrate Kit. 4. Alkaline phosphatase levels normalized to total protein content using Thermo Scientific Pierce BCA Protein Assay Kit.

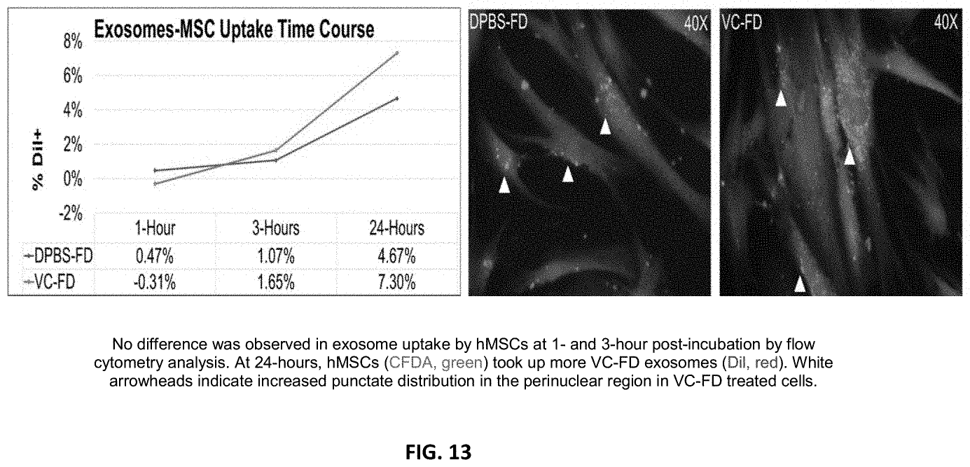

With reference to FIG. 13, hMSC Uptake Assay: 1. Lonza hMSCs were cultured and membrane stained with Carboxyfluorescein Diacetate (CFDA). 2. Exosome membranes were stained with the lipophilic tracer Dil and purified by ExoQuick (SBI) and centrifugation. 3. CFDA-stained hMSCs incubated with Dil-stained exosomes for various time points up to 24 hours. 4. Dil incorporation in hMSCs assessed by fluorescent microscopy and flow cytometry.

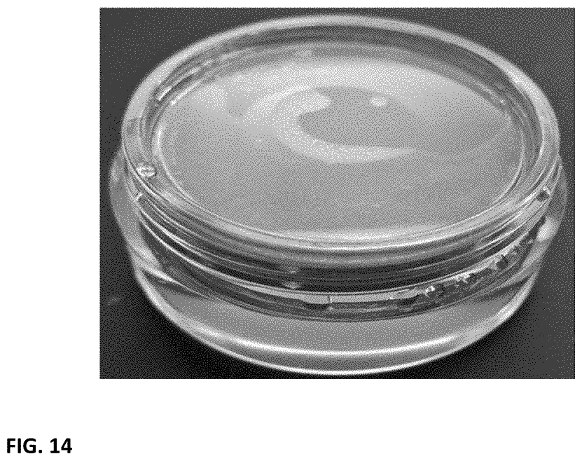

Moldable Allograft Bone Gel Infused with Spine-Derived Exosomes Triggers Osteogenic Induction is stated herein in FIGS. 14-17.

Exosomes are nanoscale vesicles that function as critical mediators of cell-to-cell communication via transportation of molecular cargo from a source cell to a target cell. It has previously been shown that the differentiation fate of primitive cells, such as mesenchymal stem cells (MSCs), can be modified towards an osteogenic path by the uptake of exosomes from defined cell types. Due to these properties, it is believed that exosomes derived from a bone source such as spine will drive the osteogenic differentiation of progenitor cells. A moldable bone gel was developed to serve as an osteoconductive support in filling bone voids. However, due to the effects of processing bone into a gelatinous material, the innate osteoinductive properties were inhibited. Therefore, the inventors sought to restore the osteoinductive capacity of the bone gel product by infusing it with spine-derived exosomes and further hypothesized osteogenic induction would be restored with this novel bone graft material.

The inventors isolated exosomes from qualified cadaveric human spines by ultrafiltration and subsequent differential ultracentrifugation (Beckman Optima XE-90 Ultracentrifuge equipped with a SW32 rotor) of the clarified supernatant. They characterized the purified exosomes by flow cytometry by coating latex beads with the nanoparticles and subsequently labeling the exosome-bound beads with known exosome markers, CD63, CD81 and CD9. Scanning Electron Microscopy (SEM) was performed to verify the size and morphology of the exosomes. Exosome concentration was determined using EXOCET Exosome Quantitation Kit (System Biosciences). Protein concentration was determined using a Qubit 4.0 Fluorometer (Thermo Fisher), from which the purity of the exosome sample was determined by calculating exosome concentration per microgram of protein. A sensitive, quantitative method to assess the bone forming potential of C2C12 myoblast cell line was used. The expression of alkaline phosphatase, a widely accepted marker for bone formation, was measured following treatment of C2C12 cells with spine-derived exosomes alone or in combination with bone gel using polycarbonate membranes, TRANSWELL®. Treatment with 50 ng of BMP-4 was used as a positive control. Alkaline phosphatase expression was normalized to total protein content, which was measured with Pierce BCA Protein Assay Kit (Thermo Fisher). The osteoinductive (OI) index was calculated by using the following formula: (OI test sample result−OI negative control result)/OI negative control result/protein concentration. An index over 20% of negative baseline was considered as osteoinductive.

Spine-derived exosomes positively expressed the exosome flow cytometry markers tested. Specifically, they expressed 99±1% of CD81, 85±14% of CD63 and 64±35% of CD9. SEM imaging revealed most of the exosomes were approximately 100 nm in size, consistent with the expected physiological size range of exosomes (30-150 nm). The mean concentration of the spine-derived exosomes obtained was 1.22±1.0⁹×10¹⁰ exosomes/mL of supernatant. The mean number of exosomes per microgram of protein was 3.31±2.33×10⁸ indicating relatively high purity. Osteoinductive testing was performed using different concentrations of exosomes either alone or in combination with bone gel. The OI index of treatment of C2C12 cells with BMP-4 or 2×10⁸, 1×10⁹, 2×10⁹, 5×10⁹ or 1×10¹⁰ exosomes alone was 28.5, 1.0, 3.7, 7.4, 11.8 and 27.6 respectively. The OI index of treatment with 2×10⁸, 1×10⁹, 2×10⁹, 5×10⁹ or 1×10¹⁰ exosomes, with each dose combined with 0.25 cc of bone gel, was 0.9, 4.5, 6.2, 9.3 and 18.5 respectively. These results revealed a dose-dependent effect, with higher doses of exosomes resulting in a greater amount of alkaline phosphatase expression. All doses were 20% above negative baseline indicating an osteoinductive effect at doses ranging from 2×10⁸ to 1×10¹⁰ exosomes alone or with bone gel. All data is expressed as mean±S.E.M. from 3 separate experiments. Statistical analysis was performed using Student's t-test or one-way ANOVA followed by Bonferroni's post hoc test if multiple group comparisons were performed.

In this study, the inventors have demonstrated the in vitro osteoinductive effect of spine-derived exosomes alone or infused in bone gel on C2C12 cells. Although they tested different concentrations of exosomes, they only tested one concentration (50 ng) of BMP-4 as a positive control. To be able to make direct comparisons of varying concentrations of exosomes with the positive control, future studies will include higher concentrations of BMP-4 to determine the saturation of alkaline phosphatase production. Future studies will also examine if the treatment will translate in vivo in a bone defect animal model. There are specific and key miRNA transcripts involved in the observed osteoinductive regulation. Future testing will continue to include miRNA analysis to better understand the molecular mechanism of exosome-delivered therapy in the context of bone regeneration.

The significance/clinical relevance of administering exosomes alone or in combination with an exogenous scaffold, such as a bone gel in this case, has the potential to repair injured tissue to restore bone function. The clinical significance of this application is aimed to promote patients' bone healing repair process and provide a cell-free therapeutic platform that is safe and effective.

Assays relating to moldable allograft bone gel include:

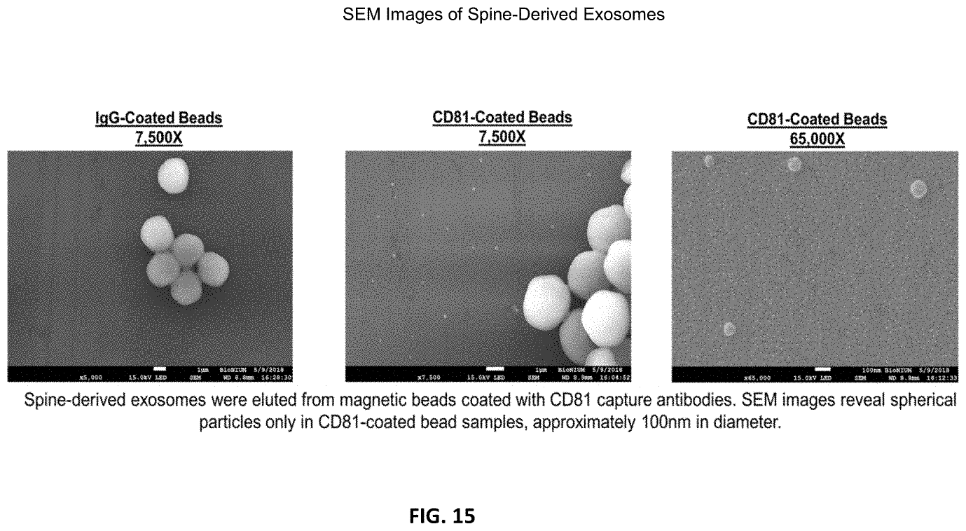

With reference to FIG. 15, Scanning Electron Microscopy: 1. Protein G magnetic beads (Bio-Rad) coated with CD81 antibodies. 2. Exosome sample incubated and immunocaptured on beads. 3. Beads washed, fixed in 3.2% glutaraldehyde and sequentially dehydrated with increasing concentration of ethanol. 4. Exosomes eluted from beads and sample allowed to dry on aluminum slide. 5. Images obtained with scanning electron microscope at 15 kV accelerating voltage.

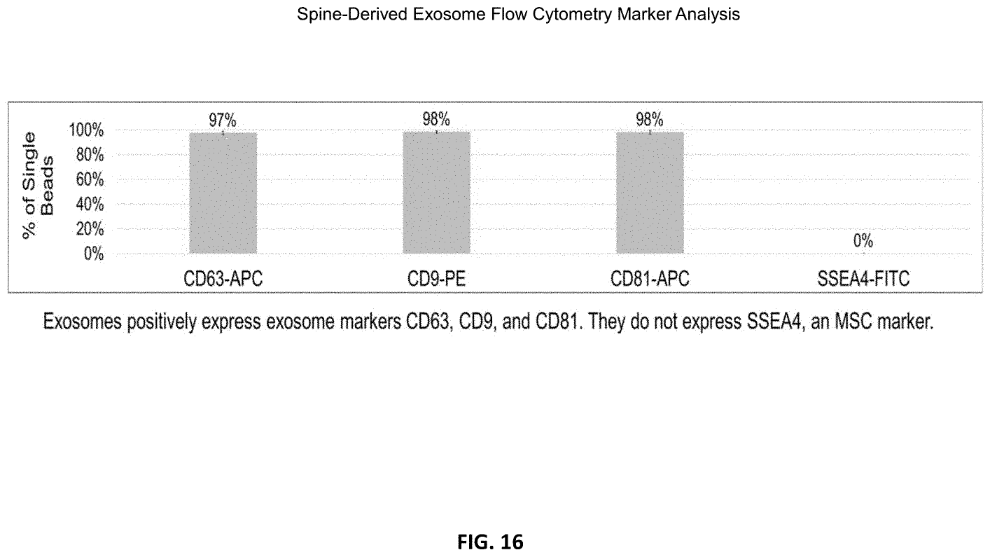

With reference to FIG. 16, Flow Cytometry with Latex Beads: 1. Aldehyde/Sulfate latex beads (4% w/v, 4 μm) coated with exosome sample. 2. Beads stained for exosomal flow markers CD63, CD81, and CD9 and MSC negative control marker SSEA-4. IgG-conjugated fluorophore antibodies used as negative control. 3. Samples analyzed by flow cytometry and single beads gated for analysis.

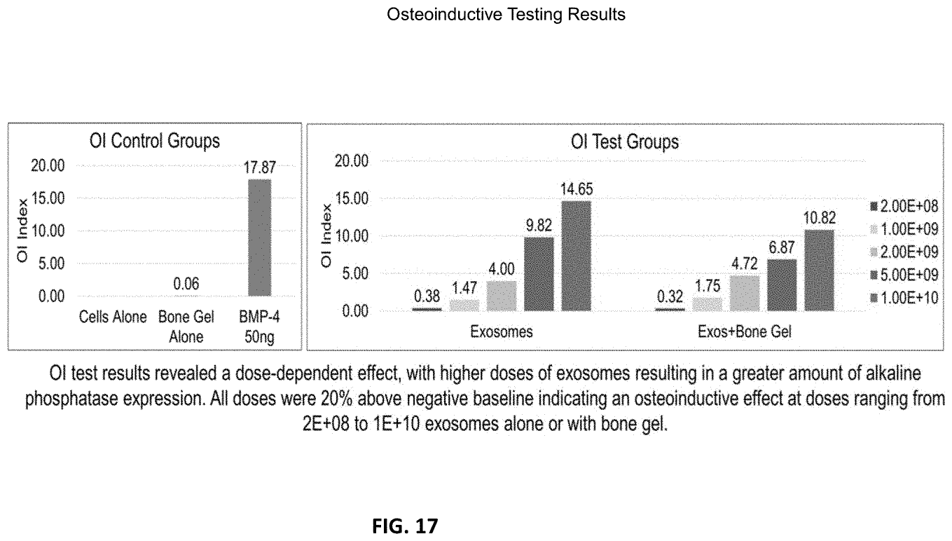

With reference to FIG. 17, Osteoinductive Testing with C2C12 (Pluripotent) Cells: 1. C2C12 cells seeded in 24-well plate. 2. Cells treated with different exosome sample types using TRANSWELL®. BMP-4 treatment used for positive control. 3. Following 48-hour exposure, cells assayed for alkaline phosphatase activity using Thermo Scientific Pierce PNPP Substrate Kit. 4. Alkaline phosphatase levels normalized to total protein content.

In embodiment, the manufacturing of a spine-derived exosome product is derived from the manufacturing process discussed above. Exosomes are small membrane vesicles (30 nm-150 nm) secreted by all cell types and naturally found in bodily fluids. They contain nucleic acids, proteins, lipids, and miRNA and have a fundamental role in cell-to-cell communication. The spine derived exosomes described herein are purified following processing of qualified cadaveric spine tissue from donors between the ages of 15-55 in accordance with FDA (21 CFR Part 1271) and to the standards of the American Association of Tissue Banks (AATB). Recovery of the donor tissue is performed according to procedures already established. The final processed spine-derived exosome product can be combined with bone matrix and/or bone gel products and is intended for homologous use as a bone void filler.

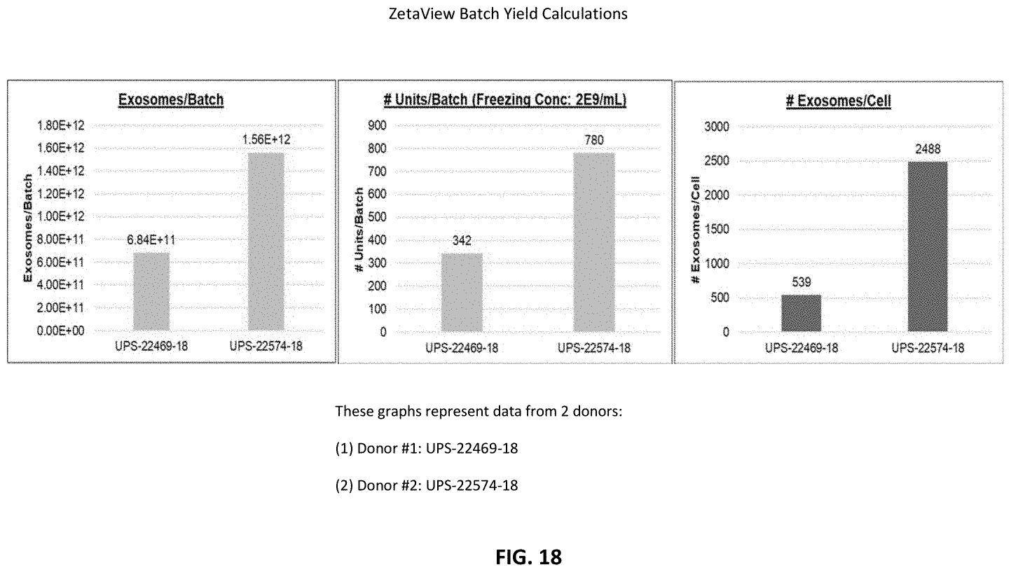

With reference to FIG. 18, an interesting aspect of the present invention is to establish an estimate of exosome batch yields. FIG. 18 shows graphs of exosomes/batch, #units/batch and exosomes/live cell. These graphs represent data from 2 donors: (1) Donor #1: UPS-22469-18 (2) Donor #2: UPS-22574-18. The chart on the left in FIG. 18 shows exosomes/batch, the middle chart shows the units/batch at a freezing concentration of 2E9/mL. The #exosomes/live cell is the third chart on the right of FIG. 18. As can be seen, the number of exosomes per unit is higher than the >1×10⁸ minimum. This is to allow for processing losses during the freeze-drying process. Previously, the inventors used a biochemical method to quantitate exosomes (EXOCET). The inventors acquired an instrument called ZETAVIEW™ which is a nanoparticle tracking analyzer (NTA). This method is more accurate. Due to the differences, the effective concentrations the inventors are using is different than what was previously reported due to the superior accuracy.

The inventors tested a variety of cryoprotectants, the COOH-epsilon PLL versions were variations of the patented Matsumura protectant of U.S. Pat. No. 9,603,355 entitled “Composition for Cryopreservation of Cells” which is incorporated herein in its entirety by reference. Some having more or less or the same carboxylation percentage.

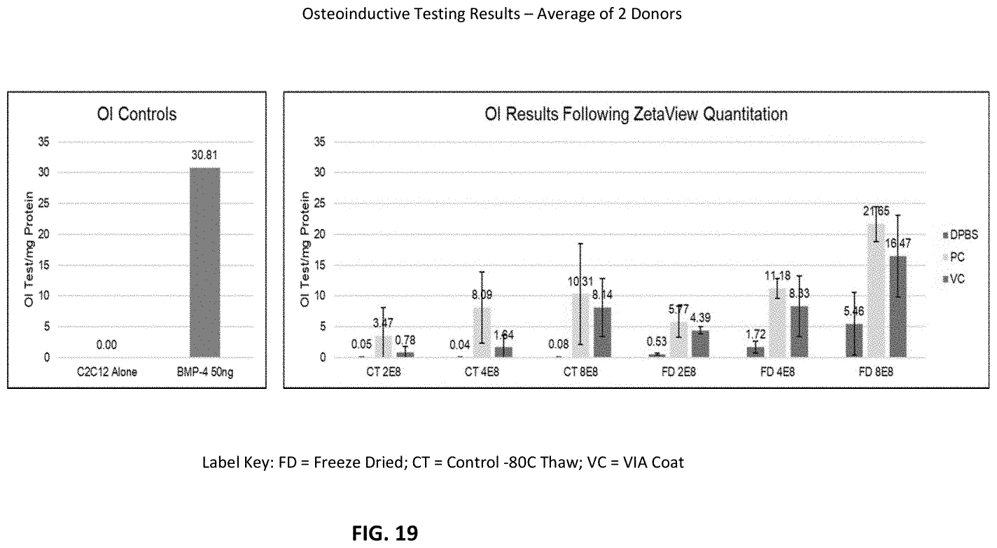

FIG. 19 shows osteoinductive (OI) testing results of the average of the two donors. FIG. 19 shows graphs of control and test groups for ZETAVIEW™ exosome titration. Label Key: FD=Freeze Dried; CT=Control −80 C Thaw; VC=VIA COAT™. Consistent with previous testing, there is a dose-dependent OI effect. Based on these results, FD 8E8 exosomes performed optimally. PC and VC are two slightly different formulations of a carboxylated ε-poly-l-lysine cryoprotectant. VC is typically a pH approximately 7.0 or neutral. PC is slightly shifted to a lower pH and slightly acidic. These attributes will be discussed later. There is no statistically significant difference between PC and VC.

In determining freezing concentration, the Concentration Range Tested=2E8 to 8E8. 8E8 performed similar to BMP-4 positive control. Due to the reduction in exosome concentration observed in VC-FD samples, the inventors calculated the % reduction and factored this into the effective concentration (based on OI results). This will ensure the post-lyophilization concentration will be close to the effective concentration to meet minimum acceptance criteria. Based on OI testing, results demonstrated passing OI criteria with exosome concentration as low as 2E8. Setting the Freezing Concentration of Final VIA COAT™ Product to equal 2E9 exosomes/mL, it is expected the final concentration to be around 8E8 after the freeze-drying process.

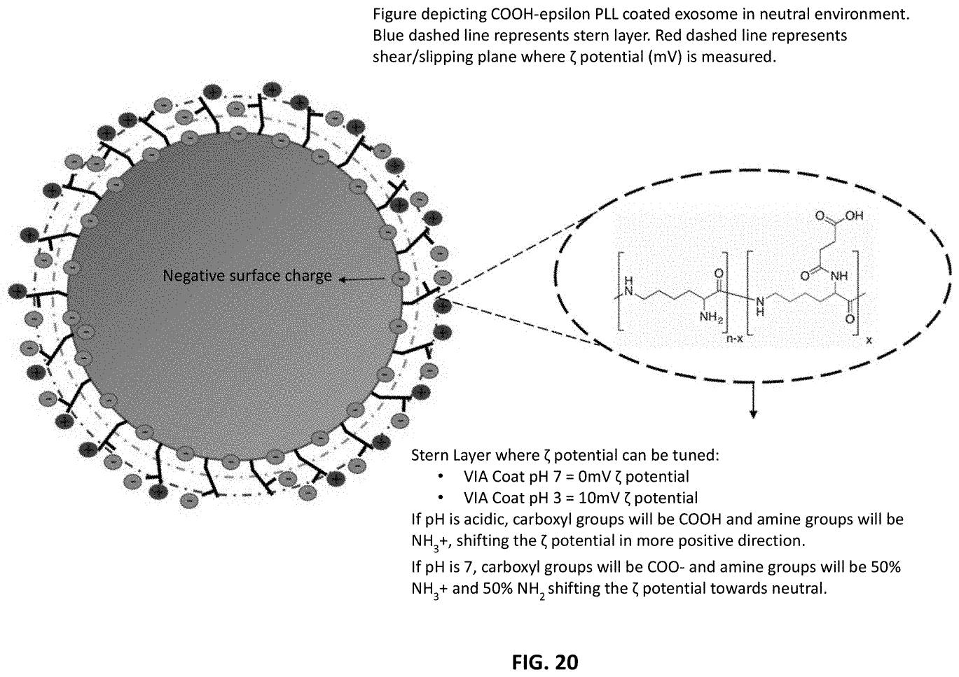

FIG. 20 is a figure depicting COOH-epsilon PLL coated exosome in neutral environment. Figure depicting COOH-epsilon PLL coated exosome in neutral environment. Blue dashed line represents stern layer. Red dashed line represents shear/slipping plane where ζ potential (mV) is measured. Stern Layer where ζ potential can be tuned by adjusting the pH of the protectant, for example: VIA COAT™ pH 7=0 mV potential; VIA COAT™ pH 3=10 mV ζ potential. If pH is acidic, carboxyl groups will be COOH and amine groups will be NH3+, shifting the ζ potential in more positive direction. If pH is 7, carboxyl groups will be COO— and amine groups will be 50% NH3+ and 50% NH2 shifting the ζ potential towards neutral.

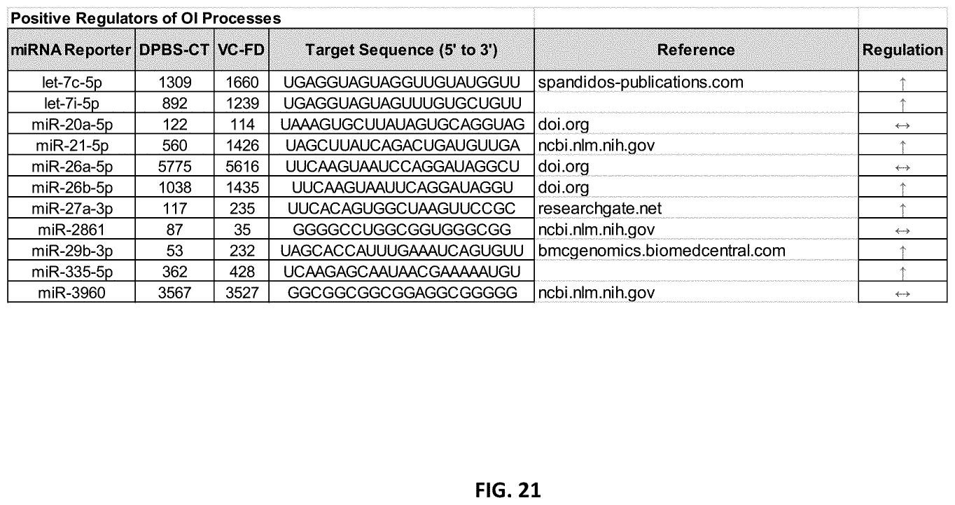

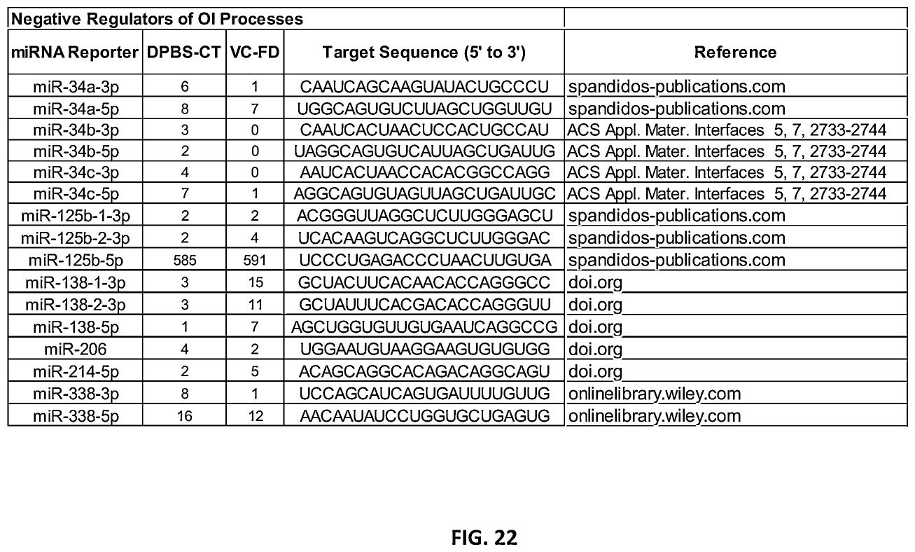

FIG. 21 is a chart of miRNA Claims—Combined Donor Data—POSITIVE Regulators of OI processes. FIG. 22 is a chart of miRNA Claims—Combined Donor Data—NEGATIVE Regulators of OI processes. Distinct miRNA profile; Regulation of essential miRNA to control biological processes such as osteogenesis and angiogenesis. Examples of negative regulators that are downregulated in our final lyophilized exosome product: miR-125, miR-214. Examples of positive regulators that are upregulated in our final lyophilized exosome product: let-7c, let-7i, miR-21, miR-26a, miR-27a, miR-335, miR-3960. Multilineage agnostic cells obtained from various satellite sources, including but not limited to bone marrow, muscle, CSF. When these cells are exposed to spine, muscle, cartilage, nerves, disc, etc., they offer the advantage of specifying miRNA content. These miRNA have been previously shown to regulate osteogenic or angiogenic processes, but not necessarily expressed in exosomes. Our data demonstrates distinct expression in spine-derived exosome fractions.

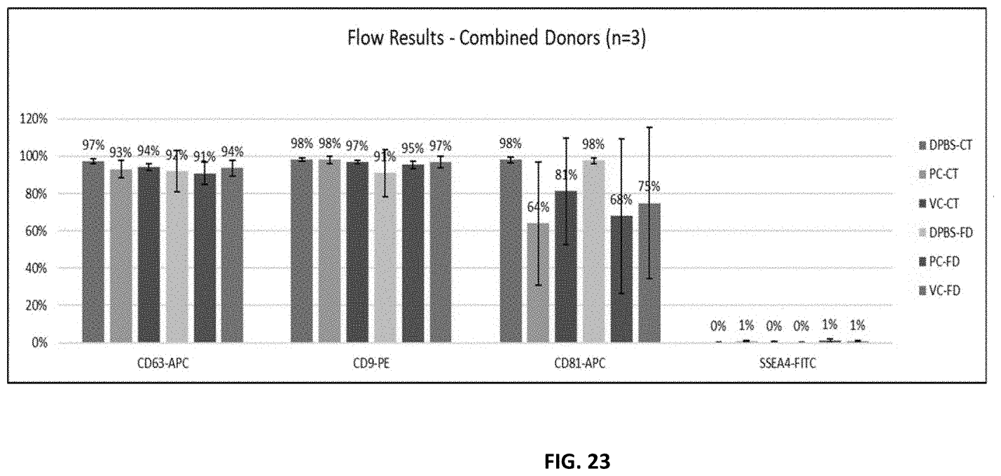

FIG. 23 is a graph of flow cytometry results—combined donors (n=3). Freeze-Drying maintains canonical exosome marker expression including CD63, CD9, CD81, indicating preservation of proteins. No statistical difference between groups.

SEQ ID NO. 1
ugagguagua gguuguaugg uu
SEQ ID NO. 2
ugagguagua guuugugcug uu
SEQ ID NO. 3
uaaagugcuu auagugcagg uag
SEQ ID NO. 4
uagcuuauca gacugauguu ga
SEQ ID NO. 5
uucaaguaau ccaggauagg cu
SEQ ID NO. 6
uucaaguaau ucaggauagg u
SEQ ID NO. 7
uucacagugg cuaaguuccg c
SEQ ID NO. 8
ggggccuggc ggugggcgg
SEQ ID NO. 9
uagcaccauu ugaaaucagu guu
SEQ ID NO. 10
ucaagagcaa uaacgaaaaa ugu
SEQ ID NO. 11
ggcggcggcg gaggcggggg
SEQ ID NO. 12
caaucagcaa guauacugcc cu
SEQ ID NO. 13
uggcaguguc uuagcugguu gu
SEQ ID NO. 14
caaucacuaa cuccacugcc au
SEQ ID NO. 15
uaggcagugu cauuagcuga uug
SEQ ID NO. 16
aaucacuaac cacacggcca gg
SEQ ID NO. 17
aggcagugua guuagcugau ugc
SEQ ID NO. 18
acggguuagg cucuugggag cu
SEQ ID NO. 19
ucacaaguca ggcucuuggg ac
SEQ ID NO. 20
ucccugagac ccuaacuugu ga
SEQ ID NO. 21
gcuacuucac aacaccaggg cc
SEQ ID NO. 22
gcuauuucac gacaccaggg uu
SEQ ID NO. 23
agcugguguu gugaaucagg ccg
SEQ ID NO. 24
uggaauguaa ggaagugugu gg
SEQ ID NO. 25
acagcaggca cagacaggca gu
SEQ ID NO. 26
uccagcauca gugauuuugu ug
SEQ ID NO. 27
aacaauaucc uggugcugag ug

The inventors have demonstrated that carboxylated epsilon PLL cryoprotectant provides stability to exosome colloidal dispersion by resisting flocculation/agglomeration effects. That electrostatic and charge stabilization can be engineered by tuning the pH or titrating polyelectrolytes or ions. One cryoprotectant tested, an acidic carboxylated epsilon PLL cryoprotectant, has a more positive (˜10 mV) zeta potential; VC or VIA COAT™ is more positive than DPBS however towards 0 mV zeta potential; NH₂ binds to negative surface charge of exosome membrane with COO— and free NH₂ groups facing outwards. All these cryoprotectants can be adjusted or tuned to achieve desired outcomes.

Steric stabilization or repulsion polymers is added to the exosomes coating onto the exosome particle surface and preventing exosome particles from coming into contact with each other. The thickness of the coating is sufficient to keep exosome particles separated by steric repulsions between the polymer layers.

Carboxylated epsilon PLL serves as a more effective cryoprotectant as well as a lyoprotectant compared to industry standard DPBS.

The cryopreserved and lyophilized exosomes in carboxylated epsilon PLL cryoprotectant demonstrated significantly enhanced regenerative capabilities.

The inventors confirmed freeze-drying maintains biological properties of exosomes. In terms of osteoinduction, results demonstrated an equivalent index compared to BMP-4 (known potent osteoinductive molecule).

Exosomes made in accordance with the present invention retain specific biological protein markers CD63, CD9 and CD81, which was also confirmed by flow cytometry.

They also found Bone Gel alone is not osteoinductive, however when combined with exosomes according to the inventors, restores OI properties, yielding a product that is osteoconductive and osteoinductive.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

What is claimed is:

1. A composition of exosomes
comprising:
a plurality of exosomes; and
a biocompatible cryoprotectant;
wherein the exosomes are suspended in the cryoprotectant creating a colloidal suspension of exosomes.

2. The composition of claim 1 wherein the plurality of exosomes is at a concentration in a suitable range of number of exosomes to be used therapeutically is about 1×10³ to about 1×10¹² exosomes/ml.

3. The composition of claim 2, wherein the quantity of exosomes is greater than 1×10⁸ exosomes/ml.

4. The composition of claim 1, wherein the colloidal suspension of exosomes is frozen at −65 degrees C. or colder, forming a frozen composition of exosomes.

5. The composition of claim 4, wherein the frozen composition of exosomes is stored at about −65 degrees C.

6. The composition of claim 1, wherein the colloidal suspension of exosomes is freeze-dried, forming a freeze-dried composition of exosomes, wherein the freeze-dried composition of exosomes is stored at ambient conditions in a vacuum sealed container.

7. The composition of claim 6, wherein the exosomes are at a concentration of 8E8 or greater.

8. The composition of claim 1, wherein the cryoprotectant is a carboxylated ε-poly-l-lysine (COOH-PLL) cryoprotectant.

9. The composition of claim 8, wherein the carboxylated ε-poly-l-lysine (COOH-PLL) provides stability to exosome dispersion by resisting flocculation or agglomeration.

10. The composition of claim 8, wherein the cryoprotectant is configured to induce an electrostatic charge stabilization.

11. The composition of claim 8, wherein the cryoprotectant is configured to yield an acidic pH, a neutral pH or a base pH to achieve positive, negative, or zero zeta potentials.

12. The composition of claim 11, wherein the composition comprises a 7.0 pH and 0 mV zeta potential.

13. The composition of claim 11, wherein the cryoprotectant yields a positive or negative mV zeta potential.

14. The composition of claim 11, wherein the cryoprotectant yields a 10 mV zeta potential.

15. The composition of claim 10, wherein the cryoprotectant comprises a steric stabilization or repulsion due to a polymer coating on the surface of at least some of the exosomes that prevent the exosomes from coming into contact with each other.

16. The composition of claim 15, wherein the coating has a thickness that is sufficient to keep particles separated by steric repulsions between the polymer layers.

17. The composition of claim 8, wherein cryopreserved and lyophilized exosomes in the carboxylated epsilon PLL cryoprotectant enhance regenerative capabilities.

18. The composition of claim 17, wherein freeze-drying maintains osteoinductive or osteoconductive properties of the exosomes.

19. The composition of claim 1, wherein the exosomes express CD63, CD9, and CD81 and MSC negative control marker SSEA-4.

20. The composition of claim 1, wherein the exosomes have inherent and varied biologic expression of miRNA that modulate biological processes such as osteogenesis, osteoglastogenesis, angiogenesis, and marrow supplementation that are not limited to germ layer and apply across a range of differentiation that would be termed pluripotent.

21. The composition of claim 20, wherein miR-125, miR-214, or both miR-125 and miR-214 are downregulated in the exosomes.

22. The composition of claim 20, wherein let-7c, let-7i, miR-21, miR-26a, miR-27a, miR-335, miR-3960, or combinations thereof are upregulated in the exosomes.

23. A kit comprising:
a) an exosome composition, comprising a quantity of exosomes and
a biocompatible cryoprotectant;
wherein the quantity of exosomes is suspended in the cryoprotectant and then frozen or freeze-dried to create an exosome composition; and
b) a quantity of one or more bone derived components or combinations thereof of a bone gel, a cortical bone, a cancellous bone, or a demineralized bone, a partially mineralized bone or a mineralized bone, or a bone material infused with cryoprotectant and exosome quantity and then subsequently lyophilized.

24. The kit of claim 23, wherein the exosome composition is combined with the one or more bone derived components or combination thereof to form an osteoinductive or osteoconductive product.

25. The kit of claim 23 wherein the exosome product is fabricated to exactly fit a bone defect and that the transfer of osteogenic, or biologic effect is intended as adjacent.

26. The kit of claim 24, wherein the bone derived components are fibers, particles, or combinations thereof.

27. A method of making a composition of exosomes, comprising:
a) creating a solution from a washed bulk tissue source submerged with at least twice the volume of prepared Processing Media with Antibiotics (PMWA) and incubating, wherein the solution contains non-whole cellular components and whole cells;
b) filtering the solution from the washed bulk tissue source;
c) separating the non-whole cellular components from the whole cells by centrifugation to form a cell pellet and a supernatant above the cell pellet;
d) ultra-centrifuging the supernatant to form a pellet of cell debris and discarding the pellet of cell debris;
e) filtering the supernatant with a sub-micron filter up to 0.5 micron; and
f) centrifuging the supernatant to form an exosome pellet.

28. The method of claim 27, further comprising:
suspending the exosome pellet in a fluid wash of Dulbecco's Phosphate Buffered Saline (DPBS);
centrifuging to form the exosome pellet and a second supernatant with unwanted proteins; and
discarding the second supernatant.

29. The method of claim 27, further comprising:
forming an exosome fluid suspension by suspending the exosome pellet in a fluid.

30. The method of claim 28, further comprising:
measuring a sample of the suspended fluid to determine the concentration of exosomes per ml. in the suspended fluid.

31. The method of claim 30, further comprising: freeze drying the exosomes in the fluid.

32. The method of claim 31, wherein the fluid is a cryoprotectant.

33. The method of claim 31, wherein the exosome fluid suspension is frozen to form a frozen exosome composition for storage.

34. The method of claim 27, wherein the incubation occurs over a duration of several hours between 18-24 hours.

35. The method of claim 26, wherein the sub-micron filter is a 0.2 micron filter.
